(19) United States
(12) Patent Application Publication
(10) Pub. No.: US 2025/0057881 A1
(43) Pub. Date: Feb. 20, 2025

(54) COMPOSITIONS AND METHODS FOR USE OF RECOMBINANT T CELL RECEPTORS AGAINST CLAUDIN 6

(71) Applicant: Roswell Park Cancer Institute Corporation, Buffalo, NY (US)

(72) Inventors: Kunle ODUNSI, Oak Park, IL (US); Junko MATSUZAKI, Munster, IN (US); Takemasa TSUJI, Munster, IN (US)

(21) Appl. No.: 18/700,698

(22) PCT Filed: Oct. 14, 2022
(86) PCT No.: PCT/US2022/078112

§ 371 Date: Apr. 11, 2024

Related U.S. Application Data
(60) Provisional application No. 63/255,786, filed on Oct. 14, 2021.

(51) Int. Cl. A61K 35/17; A61K 39/00; C07K 14/725; C12N 5/0783

(52) U.S. Cl. CPC A61K 35/17; A61K 39/4611; A61K 39/4632; A61K 39/464466; C07K 14/7051; C12N 5/0636; A61K 2239/59; C12N 2510/00

(57) ABSTRACT

Provided are compositions and methods for prophylaxis and/or therapy of a variety of cancers which express a Claudin 6 (CLDN6) antigen. Included are recombinant T cell receptors (TCRs), polynucleotides encoding them, expression vectors that include the polynucleotides, and cells into which the polynucleotides have been introduced to produce modified cells, including CD4+ T cells, CD8+ T cells, and progenitor cells, such as hematopoietic stem cells. The modified cells are capable of direct and indirect recognition of a cancer ell expressing a CLDN6 antigen by human leukocyte antigen (HLA) class I and II restricted binding of the TCR to the CLDN6 antigen expressed by the cancer cell, with or without presentation of the antigen by antigen presenting cells. Also included is a method for prophylaxis and/or therapy of cancer by administering modified cells that express a recombinant TCR that binds to CLDN6.

### CROSS REFERENCE TO RELATED APPLICATIONS

This application claims priority to U.S. provisional application No. 63/255,786, filed Oct. 14, 2021, the entire disclosure of which is incorporated herein by reference.

### SEQUENCE LISTING

The instant application contains a Sequence Listing which has been submitted in xml format and is hereby incorporated by reference in its entirety. Said.xml copy was created on Oct. 13, 2022, is named “003551_01062.xml”, and is 30,441 bytes in size.

### FIELD

The present disclosure relates generally to immunotherapy and more specifically to recombinant T cell receptors (TCR) that can impart tumor recognition capability to T cells by targeting Claudin 6 (CLDN6).

### BACKGROUND OF THE INVENTION

Tumor antigen-specific T cells recognize and kill cancer cells by using unique TCR alpha and beta chain complex which specifically binds to tumor antigen peptide/HLA complex. Extremely diverse TCR alpha/beta sequence, which is generated by random deletion and insertion of nucleotides during the recombination process, determines peptide-specificity, HLA restriction, and strength of recognition. Gene-engineering of polyclonally expanded peripheral T cells with tumor antigen-specific TCR generates large numbers of tumor antigen-specific T cells that can be used in adoptive T cell therapy (ACT) of cancer patients. ACT using gene-engineered T cells is a potent and practical therapeutic approach for cancer patients. However, eligible patients for the therapy are limited by target antigen expression and specificity of engineered T cells. Therefore, there is a critical and urgent need to find promising TCR genes that are strictly specific to tumor antigens that highly express in a broad range of patients. The present disclosure is pertinent to this need.

### SUMMARY

The present disclosure demonstrates naturally occurring T-cell response against Claudin 6 (CLDN6) in cancer patients, cloning TCR genes from CLDN6-specific T cells, and generation of CLDN6-specific TCR-engineered T cells for potential clinical use. CLDN6 expression is observed in other solid tumor such as endometrial, testicular, lung and gastric cancers but not in normal tissues. CLDN6-specific TCR genes derived from patients have never been reported. The disclosure demonstrates that CLDN6 can be targeted by engineered T cell therapy for ovarian cancer patient. Among other aspects, the disclosure demonstrates: (i) frequent and specific expression on EpCAM+ primary ovarian cancer cells in ascites fluid, (ii) CLDN6-specific T-cell responses, (iii) identification of HLA-A*02:01 and HLA-DR*04:04 epitopes, (iv) cloning and sequencing HLA-A*02:01 and HLA-DR*04:04-restricted and CLDN6-specific TCR genes, (v) generation and characterization of CLDN6-specific TCR-engineered T cells, and (vi) demonstration of cancer recognition by CLDN6-specific TCR-engineered T cells.

In one aspect the disclosure includes an expression vector encoding an alpha chain and a beta chain of a T cell receptor (TCR), wherein T cells modified to comprise the expression vector and express the alpha and beta chains of the TCR specifically recognize a CLDN6 protein epitope in an HLA context.

In embodiments, the T cells are CD4+ T cells, wherein the alpha chain comprises the sequence of SEQ ID NO:9, wherein optionally the 182nd amino acid is

Cysteine, and the beta chain comprises the sequence of SEQ ID NO:11, wherein optionally the 192nd amino acid is Cysteine; or wherein the alpha chain comprises the sequence of SEQ ID NO: 13, wherein optionally the 179th amino acid is Cysteine, and a beta chain having the sequence of SEQ ID NO:15, wherein optionally the 188th amino acid is Cysteine. In embodiments, the T cells are CD8+ T cells, wherein the alpha chain

comprises the sequence of SEQ ID NO: 13, wherein optionally the 179th amino acid is Cysteine, and wherein the beta chain comprises the sequence of SEQ ID NO:15, wherein optionally the 188th amino acid is Cysteine. In embodiments, the alpha chain, the beta chain, or both of the expression vector comprise said Cysteine. In one aspect the disclosure includes a hematopoietic stem cell or a T cell comprising an expression vector.

In another aspect the disclosure includes a method comprising administering to an individual who has a CLDN6 positive cancer a population of hematopoietic stem cells or T cells to thereby inhibit the growth of the cancer and/or kill the cancer cells. In embodiments, the population of T cells comprises CD8+ T cells, CD4+ T cells, or a combination thereof. In one aspect, the disclosure includes a method of making a modified T cell or a modified hematopoietic stem cell, the method comprising introducing into a T cell or a hematopoietic stem cell an expression vector to thereby produce the modified T cell or the modified hematopoietic stem cell.

### BRIEF DESCRIPTION OF THE FIGURES

FIG. 1. CLDN6 expression on EpCAM⁺ cancer cells and CD45⁺ lymphocytes in ascites fluids of ovarian cancer patients. (A) Flow cytometry analysis of ascites mononuclear cells. CLDN6 expression was analyzed on EpCAM⁺ or CD45⁺ cells. Gating of CLDN6 was determined based on FMO controls. (B) Percentages of CLDN6 expression on EpCAM⁺ or CD45⁺ cells from 32 patients were plotted with mean±standard deviation.

FIG. 2. Analysis of CLDN6-specific T-cell response in ovarian cancer patients. (A) CLDN6- or CEFT-specific T-cell responses from 17 patients (P01-P17) were determined by ELISPOT assay. PBMC were cultured with CLDN6 or CEFT peptides for 13-15 days and IFN-γ spots in the presence (pep) or absence (−) of the respective peptide were enumerated. (B) IFN-γ and TNF-α production on CD8⁺ or CD4⁺ T cells from P01 against CLDN6 peptide-pulsed or-unpulsed autologous EBV-B cells was analyzed by intracellular cytokine staining.

FIG. 3. Establishment of CLDN6-specific TCR gene-engineered T cells. Healthy donor PBMC were retrovirally transduced with TCR genes from CLDN6-specific CD8⁺ T cells (CD8-TCR) or CD4⁺ T cells (CD4-TCR). (A) Transduction efficiency of TCR- or mock-transduced T cells were analyzed by TCR-Vβ8 or -Vβ7.1 antibody. (B) IFN-γ production against CLDN6 peptide-pulsed or-unpulsed autologous EBV-B cells were analyzed by intracellular cytokine staining.

FIG. 4. Characterization of CLDN6-specific TCR gene-engineered T cells. (A) TCR gene-engineered T cells were cocultured with 20-mer CLDN6 overlapping peptides-pulsed or-unpulsed autologous EBV-B cells for 24 hours. IFN-γ level in the culture supernatant was measured by ELISA. (B) Cytokine production from CD8-TCR-engineered CD8⁺ T cells against CLDN6 peptide-pulsed K562 cells transduced with indicated HLA genes was analyzed by intracellular cytokine staining. (C) CD8-TCR-or mock-transduced T cells were stained with or without HLA-A2/CLDN6_{132- 140} tetramer. (D) IFN-γ production on CD8-TCR-transduced CD8⁺ T cells against different concentration of CLDN6₁₃₂₋₁₄₀ was analyzed by intracellular cytokine staining. Error bars indicate standard deviation of technical duplicates. (E) IFN-γ production on CD4-TCR-transduced CD4⁺ T cells stimulated with CLDN6₁₋₂₀-pulsed or-unpulsed partially HLA-matched EBV-B cells (Table 1) was analyzed by intracellular cytokine staining.

FIG. 5. Cancer cell recognition by CLDN6-specific TCR gene-engineered T cells. (A) CLDN6 and HLA-A2 expression on ovarian cancer cells with or without IFN-γ treatment was determined by flow cytometry. Expression was shown as quadrant gating based on the unstained control (left) and histogram including unstained controls (right). PA-1/A2: PA-1 transduced with HLA-A2. (B, C) CLDN6-specific CD8⁺ T-cell line (B), or CD8-TCR-or mock-transduced T cells (C) were cocultured with or without IFN-γ-treated or-untreated cancer cells and IFN-γ level in the culture supernatant was measured by ELISA. Error bars indicate standard deviation of technical duplicates.

FIG. 6. DNA finger printing of TCR inserts. (A) Schematic representation of the assay. Variable regions of TCR β and α chains were PCR amplified by colony PCR using primer pairs A+B and C+D, respectively, from E. coli clones. PCR products were digested by two restriction enzymes (AluI and MspI), and analyzed by gel electrophoresis. (B) Digestion patterns of TCR β and α variable regions of sorted T cells. For CD8+ T cells, 9/10 of TCR β, and 10/10 of TCR α showed the same digestion pattern. For CD4+ T cells, 14/16 of TCR β, and 14/16 of TCR α showed the same digestion pattern.

FIG. 7. CLDN6-specific CD8+ T-cell line. (A) TCR-Vβ8 expression of CLDN6-specific CD8+ T-cell line was analyzed by flow cytometry. (B) IFN-γ and CD107 expression on CD8+ T cells stimulated with CLDN6 peptide-pulsed or-unpulsed was analyzed by flow cytometry.

### DESCRIPTION OF THE DISCLOSURE

Unless defined otherwise, all technical and scientific terms used herein have the same meaning as commonly understood by one of ordinary skill in the art to which this invention belongs.

Every numerical range given throughout this specification includes its upper and lower values, as well as every narrower numerical range that falls within it, as if such narrower numerical ranges were all expressly written herein.

As used in the specification and the appended claims, the singular forms “a” “and” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about” it will be understood that the particular value forms another embodiment. The term “about” in relation to a numerical value is optional and means for example +/−10%.

This disclosure includes every amino acid sequence described herein and all nucleotide sequences encoding the amino acid sequences. Every antibody sequence and antigen binding fragments of them are included. Polynucleotide and amino acid sequences having from 80-99% similarity, inclusive, and including and all numbers and ranges of numbers there between, with the sequences provided here are included in the invention. All of the amino acid sequences described herein can include amino acid substitutions, such as conservative substitutions, that do not adversely affect the function of the protein that comprises the amino acid sequences.

The present disclosure relates to expression vectors encoding TCR genes for genetically modifying immune cells, including but not necessarily limited to T cells, to provide capability of recognition of CLDN6, a tumor antigen. In embodiments, the immune cells are CD4⁺ T cells, CD8⁺ T cells, or their progenitor/hematopoietic cells. The present disclosure further provides a method of inhibiting cancer growth by administering a population of hematopoietic stem cells or T cells that have been modified as further described herein to an individual who has a CLDN6 positive cancer.

Adoptive cell therapy (ACT) using tumor antigen-specific T cells is a powerful strategy for treatment of cancer patients as infusion of a large number of anti-tumor effector T cells provides immediate tumor-debulking effects. Furthermore, a subset of infused T cells is expected to differentiate into memory T cells to provide long-term immunosurveillance and potentially prevent recurrence of disease. Recent gene engineering techniques to transduce target antigen-specific chimeric antigen receptor (CAR) or T-cell receptor (TCR) enable rapid generation of a large number of autologous therapeutic T cells with potent anti-tumor activity. In our previous studies, we have cloned and characterized NY-ESO-1-specific and MHC class I-and class II-restricted TCR genes that are currently tested in a phase I clinical trial (ClinicalTrials.gov Identifier: NCT03691376). Although the safety and efficacy of ACT using NY-ESO-1-specific TCR-transduced cells in cancer patients has been demonstrated, eligible patients for the therapy are limited by NY-ESO-1 expression (30-80% of solid tumors) and patients' HLA types. In addition, NY-ESO-1 frequently shows heterogenous expression within a tumor, which allows antigen-negative tumor variants to escape immune attack. To develop broadly applicable and more efficient TCR gene-engineered T-cell therapy for cancer patients, identification of a panel of off-the-shelf TCR genes for shared cancer antigens or shared neoantigens is essential. In this regard, CLDN6 is a cell surface membrane protein expressed on multiple solid tumor tissues such as ovarian cancer, testicular cancer, and endometrial cancer while the expression is not observed on normal adult tissues at transcriptome and protein levels. Thus, CLDN6 is considered to be a promising target for antibody-based cancer immunotherapy. Because of the tumor-specific cell surface expression, CLDN6 is also a potential target for other immunotherapies such as cancer vaccines. Although the expression of CLDN6 on solid tumor has been broadly investigated, its immunogenicity in cancer patients has not been evaluated. In this disclosure, we analyzed spontaneously induced T-cell response against CLDN6 in ovarian cancer patients. By characterizing CLDN6-specific CD8⁺ and CD4⁺ T cells, we have identified novel CLDN6-derived short peptides that bind on HLA-A*02:01 (A2) and -DR*04:04. Furthermore, we have cloned HLA-A2 and -DR*04:04-restricted CLDN6-specific TCR genes and characterized cancer cell recognition of TCR gene-transduced T cells for the development of TCR-engineered T-cell therapy.

In various embodiments, the present invention provides isolated and/or recombinant polynucleotides encoding particular TCR polypeptides, cells engineered to express the TCR polypeptides, pharmaceutical formulations comprising cells which express the TCR polypeptides, and methods of using the pharmaceutical formulations to achieve a prophylactic and/or therapeutic effect against cancer in a subject. In certain embodiments, the disclosure provides mixtures of cells expressing TCRs, or cells expressing more than one TCR described herein, that are specific for distinct cancer antigens, thus presenting cell populations that can be considered polyvalent with respect to the TCRs. As used in this disclosure, a “recombinant TCR” means a TCR that is expressed from a polynucleotide that was introduced into the cell, meaning prior to the introduction of the polynucleotide the TCR was not encoded by a chromosomal sequence in the cell. The TCRs provided by the invention are capable of recognizing an epitope on

CLDN6. A representative amino acid sequence of CLDN6 is:

(SEQ ID NO: 17)
MASAGMQILGVVLTLLGWVNGLVSCALPMWKVTAFIGNSIVVAQVVWEG
LWMSCVVQSTGQMQCKVYDSLLALPQDLQAARALCVIALLVALFGLLVY
LAGAKCTTCVEEKDSKARLVLTSGIVFVISGVLTLIPVCWTAHAIIRDF
YNPLVAEAQKRELGASLYLGWAASGLLLLGGGLLCCTCPSGGSQGPSHY
MARYSTSAPAISRGPSEYPTKNYV.

TCR alpha and beta chain amino acid sequences are provides as SEQ ID Nos. 9-16. As described above, in certain embodiments, the cells provided by the invention are engineered T cells that are capable of recognizing a CLDN6 antigen via TCRs which interact with the antigen in association with HLA class I and/or HLA class II molecules.

The invention includes each and every polynucleotide sequence that encodes one or more TCR polypeptides of the invention and disclosed herein, including DNA and RNA sequences, and including isolated and/or recombinant polynucleotides comprising and/or consisting of such sequences. The invention also includes cells which comprise the recombinant polynucleotides. The cells can be isolated cells, cells grown and/or expanded and/or maintained in culture. Prokaryotic cell cultures can be used, for example, to propagate or amplify the TCR expression vectors of the invention. In embodiments, the cells can comprise packaging plasmids, which, for example, provide some or all of the proteins used for transcription and packaging of an RNA copy of the expression construct into recombinant viral particles, such as pseudoviral particles. In embodiments, the expression vectors are transiently or stably introduced into cells. In embodiments, the expression vectors are integrated into the chromosome of cells used for their production. In embodiments, polynucleotides encoding the TCRs which are introduced into cells by way of an expression vector, such as within a viral particle, are integrated into one or more chromosomes of the cells. Such cells can be used for propagation, or they can be cells that are used for therapeutic and/or prophylactic approaches.

Expression vectors for use with embodiments of this disclosure can be any suitable expression vector. In embodiments, the expression vector comprises a modified viral polynucleotide, such as from an adenovirus, a herpesvirus, or a retrovirus, such as a lentiviral vector. In an embodiment, an oncolytic viral vector is used. The expression vector is not restricted to recombinant viruses and includes non-viral vectors such as DNA plasmids and in vitro transcribed mRNA. In embodiments, a recombinant adeno-associated virus (AAV) vector may be used. In certain embodiments, the expression vector is a self-complementary adeno-associated virus (scAAV).

With respect to the polypeptides that are encoded by the polynucleotides described above, in certain aspects the invention provides functional TCRs which comprises a TCR α and a TCR ß chain, wherein the two chains are present in a physical association with one another (e.g., in a complex) and are non-covalently joined to one another, or wherein the two chains are distinct polypeptides but are covalently joined to one another, such as by a disulfide or other covalent linkage that is not a peptide bond. Other suitable linkages can comprise, for example, substituted or unsubstituted polyalkylene glycol, and combinations of ethylene glycol and propylene glycol in the form of, for example, copolymers. In other embodiments, two polypeptides that constitute the TCR α and a TCR β chain can both be included in a single polypeptide, such as a fusion protein, but separated by, for example, a ribosomal skipping sequence, or a self-cleaving peptide sequence. In embodiments, TCR α and a TCR β chain are both present in a single intact polypeptide, wherein the two chains are separated by a flexible, non-cleavable amino acid sequence, representative examples of which are known in the art and general comprise a Glycine rich linker. In non-limiting embodiments, the linker comprises Glycine and Serine. In an embodiment, the linker comprises 1-12 amino acids. In an embodiment, the linker comprises or consists of a GSG sequence. In embodiments, more than one linker can be used.

In certain embodiments, the fusion protein comprises a TCR α chain amino acid sequence and a TCR β chain amino acid sequence that have been translated from the same open reading frame (ORF), or distinct ORFs, or an ORF that contain a signal that results in non-continuous translation. In one embodiment, the ORF comprises a P2A-mediated translation skipping site positioned between the TCR α and TCR β chain. Constructs for making P2A containing proteins (also referred to as 2A Peptide-Linked multicistronic vectors) are known in the art. (See, for example, Gene Transfer: Delivery and Expression of DNA and RNA, A Laboratory Manual, (2007), Friedman et al., International Standard Book Number (ISBN) 978-087969765-5. Briefly, 2A peptide sequences, when included between coding regions, allow for stoichiometric production of discrete protein products within a single vector through a novel cleavage event that occurs in the 2A peptide sequence. 2A peptide sequences are generally short sequence comprising 18-22 amino acids and can comprise distinct amino-terminal sequences. Thus, in one embodiment, a fusion protein of the invention includes a P2A amino acid sequence. In embodiments, a fusion protein of the invention can comprise a linker sequence between the TCR α and TCR β chains. In certain embodiments, the linker sequence can comprise a GSG (Gly-Ser-Gly) linker or an SGSG (Ser-Gly-Ser-Gly) linker. In certain embodiments, the TCR α and TCR β chains are connected to one another by an amino acid sequence that comprises a furin protease recognition site, such as an RAKR (Arg-Ala-Lys-Arg) site. In embodiments, artificial cysteines are introduced to enhance pairing of transgenic alpha and beta chains.

In one embodiment, the expression construct that encodes the TCR can also encode additional polynucleotides. The additional polynucleotide can be such that it enables identification of TCR expressing cells, such as by encoding a detectable marker, such as a fluorescent or luminescent protein. The additional polynucleotide can be such that it encodes an element that allows for selective elimination of TCR expressing cells, such as thymidine kinase gene. In embodiments the additional polynucleotides can be such that they facilitate inhibition of expression of endogenously encoded TCRs. In an embodiment, the expression construct that encodes the TCR also encodes a polynucleotide which can facilitate RNAi-mediated down-regulation of one or more endogenous TCRs For example, see Okamoto S, et al. (2009) Cancer Research, 69:9003-9011, and Okamoto S, et al. (2012). Molecular Therapy-Nucleic Acids, 1, e63. In an embodiment, the expression construct that encodes the TCR can encode an shRNA or an siRNA targeted to an endogenously encoded TCR. In an alternative embodiment, a second, distinct expression construct that encodes the polynucleotide for use in downregulating endogenous TCR production can be used.

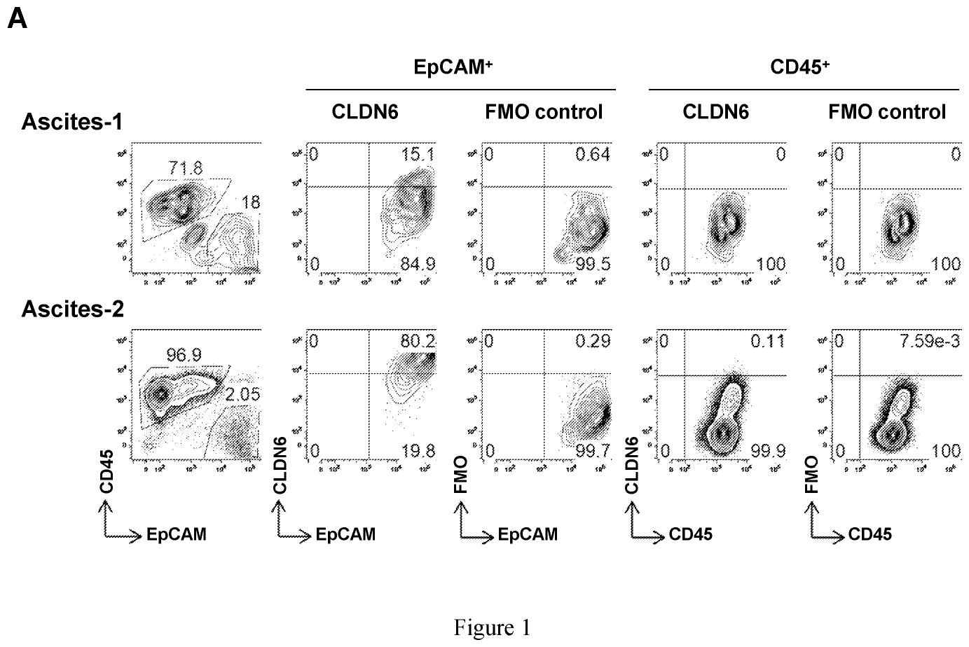

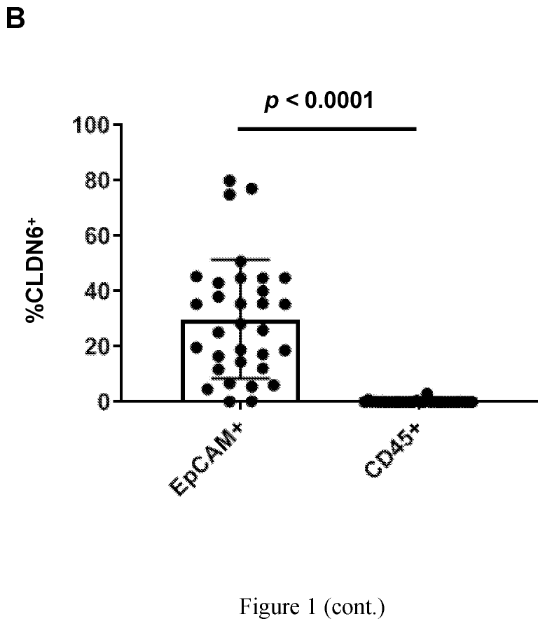

In connection with the present invention, we have also made the following discoveries: CLDN6 is an oncofetal protein expressed on ES cells associated with epithelialization as well as solid cancer cells including ovarian cancer. Flow cytometry analysis of ascites demonstrated that the majority (95%) of patients' EpCAM⁺ cancer cells express CLDN6 at varied levels (FIG. 1). Expression was low (<10%) in 13%, moderate (10-40%) in 50% and strong (>40%) in 31% of 32 samples. CLDN6 expression was observed in 69.4% of ovarian carcinomas and in 34.6% of ovarian serous adenomas by IHC. Different sensitivity of IHC and flow cytometry can explain higher expression rate in this study. Moreover, CLDN6 expression is reported at a frequency of 7% to 100% in other solid tumors such as lung, endometrial, gastric and testicular cancers.

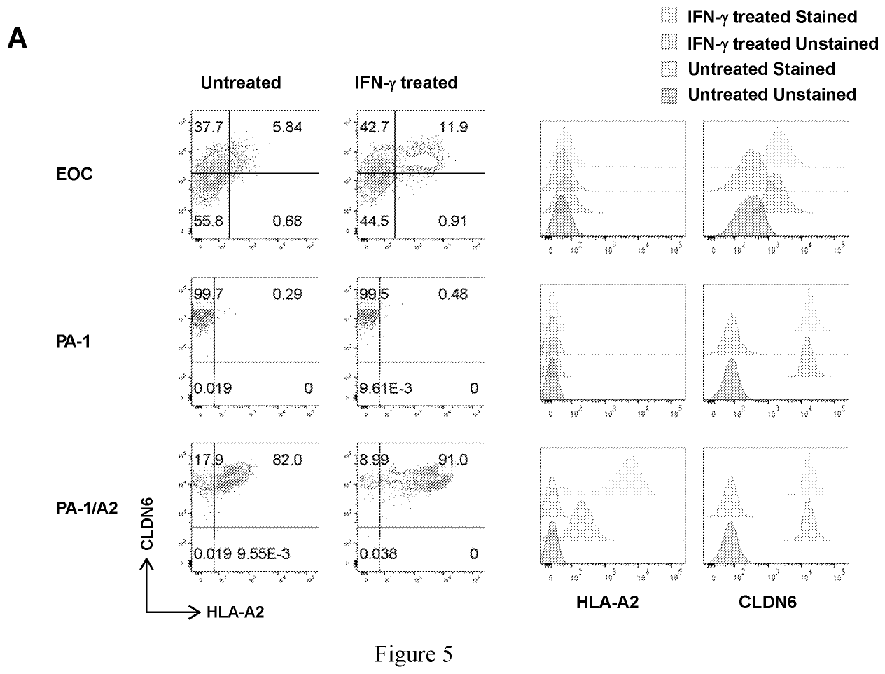

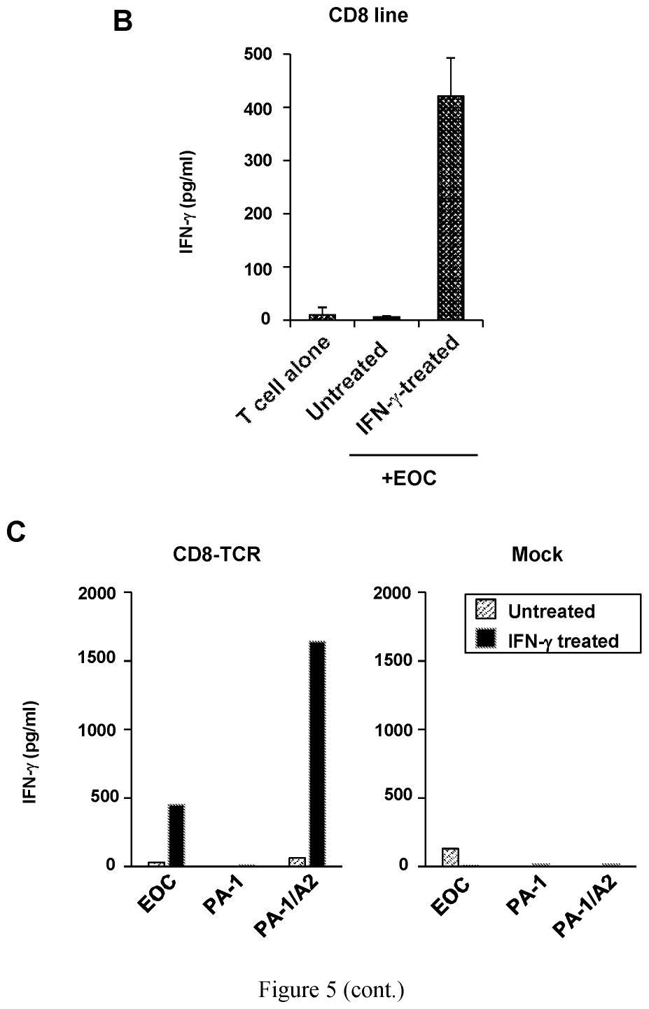

Despite the frequent expression, spontaneous T-cell response against CLDN6in peripheral blood was detected in only 1 out of 17 (6%) ovarian cancer patients. The patient (P01) who showed spontaneous anti-CLDN6 T-cell response had tumor that expressed high level of CLDN6 (FIG. 5). Without intending to be bound by any particular theory, it is considered possible that a high level of CLDN6 expression is required to induce spontaneous T-cell response.

Limited induction of T-cell response against CLDN6 in cancer patients potentially indicates low immunogenicity of the antigen, which can be overcome by ACT of genetically engineered CLDN6-specific T cells, such as those encompassed by this disclosure. As CLDN6 is expressed on cell surface of cancer cells, it is a promising target for antibody-based and CAR-T therapies. An early phase clinical trial testing CLDN6-specific CAR-T cells for solid tumor is ongoing [ClinicalTrials.gov Identifier: NCT04503278]. CLDN6-specific CAR-T cells could be a promising therapy for cancer patients with solid tumor including ovarian cancer because of frequent antigen expression.

In comparison with CAR-T cells, TCR-engineered T cells have several potential advantages including low immunogenicity of transgenes and ability to recognize antigens that are naturally processed and presented by antigen-presenting cells, both of which are considered to be critical for long-term in vivo maintenance of engineered T cells.

With respect to use of the expression vector(s) encoding TCR of the present invention, the method generally comprises administering an effective amount (typically 10¹⁰ cells by intravenous or intraperitoneal injections) of a composition comprising the hematopoietic stem cells or T cells to an individual in need thereof. An individual in need thereof, in various embodiments, is an individual who has or is suspected of having, or is at risk for developing a cancer which is characterized by malignant cells that express CLDN6. In particular and non-limiting examples, such cancers include cancers of the bladder, brain, breast, ovary, non-small cell lung cancer, myeloma, prostate, sarcoma and melanoma. The individual may have early-stage or advanced forms of any of these cancers, or may be in remission from any of these cancers. In one embodiment, the individual to whom a composition of the invention is administered is at risk for recurrence for any cancer type that expresses CLDN6. In certain embodiments, the individual has or is suspected of having, or is at risk for developing or recurrence of a tumor comprising cells which express a CLDN6.

The present disclosure includes recombinant TCRs, cells expressing them, and therapeutic/prophylactic methods that involve presentation of CLDN6 antigens/epitopes in conjunction with any HLA-class I or II complex that will be recognized by the TCRs. In embodiments, the CLDN6 antigen is recognized by the TCR in conjunction with HLA-A*02:01 (A2) and -DR*04:04. The CD8+, class I restricted TCR is restricted to HLA-A*02:01 (A2) and can directly recognize CLDN6+ cancer cells. We demonstrated that CD8+ T cells can indirectly recognize CLDN6-derived long (20-mer) peptide which was processed and presented as the short 9-mer CLDN6₁₃₂₋₁₄₀ epitope by antigen-presenting cells. The CD4+, class II restricted TCRs is restricted to HLA-DR*04:04. We demonstrated that CD4-TCR recognize CLDN6-peptide when processed by antigen-presenting cells.

We identified TCR genes specific to CLDN6 from both CD8⁺ and CD4⁺ T cells and demonstrated specific reactivity to CLDN6⁺ cancer cells by TCR-transduced T cells. We also identified HLA-A2-binding epitope as CLDN6₁₃₂₋₁₄₀ and CD8-TCR-transduced T cells were stained with the tetramer. The described CLDN6₁₃₂₋₁₄₀ epitope has the sequence TLIPVCWTA (SEQ ID NO:18). These CLDN6-specific TCR genes are considered to be safe because they spontaneously occurred and expanded in a patient. In addition, the CD8-TCR is restricted by HLA-A2 which is the most frequent HLA class I allele in the Caucasian population. Thus, this TCR could be used as an off-the-shelf therapeutic TCR gene for clinical use. A CD8-TCR described herein was found to be CD8 dependent because only TCR-transduced CD8⁺ T cells but not CD4⁺ T cells showed the reactivity against a relevant peptide. Upregulation of HLA expression on cancer cells by IFN-γ treatment was used for recognition. T cells with higher affinity TCR could be identified in patients or affinity enhancement by gene modification. We identified HLA-DR*0404-binding epitope as CLDN6₁₋₂₀ (Sequence: MASAGMQILGVVLTLLGWVN (SEQ ID NO:19). In contrast to CD8-TCR, CD4-TCR is considered as high avidity because the response was observed in a CD4 coreceptor-independent manner. The frequency of HLA-DR*04:04 is about 6% of the Caucasian population in the US.

CD8-TCR-transduced T cells showed significant in vivo reactivity against CLDN6⁺ ovarian cancer cells (FIG. 5). Thus, the data presented herein strongly suggest that the T cells targeting CLDN6 help the anti-tumor immune responses, and accordingly will likely make an effective and heretofore unavailable therapeutic approach for widespread use in the clinic.

The following description provides illustrative examples of materials and methods used to make and use various embodiments of the invention.

### Biospecimen Processing

Blood and malignant ascites were obtained from ovarian cancer patients under an approved protocol (I 215512) from the institutional review board at Roswell Park Comprehensive Cancer Center (Roswell Park). Healthy donor blood was obtained from the blood donor center at Roswell Park. Peripheral blood mononuclear cells (PBMC) and ascites mononuclear cells were isolated using lymphocyte separation media (Corning), cryopreserved in 10% DMSO/90% FBS and stored in a liquid nitrogen freezer.

### ELISPOT Assay

PBMC were thawed and 5-10×10⁵ cells were cultured with a pool of synthetic overlapping peptides for CLDN6 (1 μg/ml, JPT Peptide Technologies), or a pool of 27 peptides of Cytomegalovirus, Epstein-Barr virus, Influenza virus and Tetanus toxin (CEFT; 0.5 μM/each, GenScript) in RPMI1640 medium (Corning) supplemented with 10% human AB serum (Gemini), 2 mM L-glutamine (Corning), 1× MEM non-essential amino acid (Corning), 100 U/ml penicillin (Corning), 100 μg/ml streptomycin (Corning), 10 U/ml IL-2 (Roche) and 10 ng/ml IL-7 (R&D Systems) in 96-well round-bottom plates. The cells were expanded every 3-4 days. After 13-15 days of culture, cells were harvested and suspended in X-VIVO 15 media (Lonza). Fifty thousand cells were seeded in ELISPOT plate (MultiScreen-HA filter plate, Millipore Sigma) that were pre-coated with anti-IFN-γ antibody (clone 1-DIK, Mabtech) in the presence or absence of the respective peptide mix. The plate was incubated in 5% CO2 incubator for 20-24 hours, followed by development with biotinylated detection anti-IFN-γ antibody (clone 7-B6-1, Mabtech), streptavidin-ALP (Mabtech) and BCIP/NBT substrate (Sigma-Aldrich). The number of spots were counted using ImmunoSpot S6 Core analyzer (CTL). The response was considered as positive when the number of spots is ≥50 and twice higher than background.

### CLDN6 Expression

Mononuclear cells from ascites were thawed and stained with Fixable Viability Stain 700 (BD Horizon) followed by antibodies against CD45 (clone H130, BioLegend), CD326 (EpCAM; clone 9C4, BioLegend), and CLDN6 (clone 342927, R&D Systems). PA-1 ovarian cancer cell line was obtained from ATCC. Epithelial ovarian cancer (EOC) cell line was established from solid tumor single cell suspension. Cancer cell lines were cultured in the presence or absence of 1,000 U/ml human IFN-γ recombinant protein (PeproTech) for 2 days. The cells were harvested using 0.25% trypsin-EDTA solution (Corning) and stained with antibodies for HLA-A2 (clone BB7.2, BioLegend) and CLDN6. Cells were acquired by BD Fortessa and analyzed using FlowJo software.

### Generation of CLDN6-Transduced T Cells

CLDN6-specific T cells were isolated as previously described using IFN-γ secretion assay kit (Miltenyi Biotech. Briefly, T cells presensitized with CLDN6 peptide were incubated with CLDN6 peptide-pulsed autologous EBV-transformed B (EBV-B) cells for 4 hours and stained with IFN-γ secretion assay kit. IFN-γ⁺ cells were sorted using BD FACSAria cell sorter (BD Biosciences). One thousand cells were used for RT-PCR amplification of TCR α and β chain genes that were assembled to generate TCR-expressing plasmid vectors as previously described. Culture supernatant from high-titer retrovirus producing PG13 clone was harvested and infected to healthy donor PBMC. Transduction efficiency was determined by flow cytometry using anti-Vβ8 (for CD8-TCR; clone JR2, BioLegend) and anti-Vβ7.1 (for CD4-TCR; clone ZOE, Beckman Coulter) antibodies.

TABLE 1
HLA class II allele of patient and EBV-B cells. HLA allele
matched with P01 is shown as bold and underlined.

| Sample | HLA-DR | | HLA-DQ | | HLA-DP | |
|---|---|---|---|---|---|---|
| P01 | *0404 | *1401 | *0302 | *0503 | *0201 | *0401 |
| EBVB-1 | *1102 | *1401 | *0301 | *0503 | *0201 | |
| EBVB-2 | *0301 | *0701 | *0201 | *0303 | *0401 | |
| EBVB-3 | *04 | *07 | *0201 | | *0401 | |
| EBVB-4 | *0404 | *1101 | *0301 | *0302 | *0401 | |
| EBVB-5 | *0401 | *1101 | *0301 | *0302 | *0401 | *0402 |
| EBVB-6 | *1101 | *1301 | *03 | *06 | *0201 | *0401 |

### T-Cell Functional Analysis

CLDN6-specific CD8⁺ T-cell line was established by expanding sorted CLDN6-specific T cells with 10 μg/ml phytohemagglutinin in the presence of 30Gy γ-irradiated healthy donor PBMC, 10 U/ml IL-2 and 10 ng/ml IL-7. For intracellular cytokine staining, T cells were stimulated with peptide-pulsed or-unpulsed autologous or HLA-matched EBV-B cells (Table 1) for 6 hours in the presence of 5 μg/ml monensin (Sigma-Aldrich). In some experiments, anti-CD107a (clone H4A3, BD Biosciences) and anti-CD107b (clone H4B4, BD Biosciences) antibodies along with 5 μg/ml Brefeldin A (Sigma-Aldrich) were added during incubation. Cells were stained for CD3 (clone OKT3,BioLegend), CD4 (clone OKT4, BioLegend) and CD8 (clone RPA-T8, BioLegend), followed by fixation with 2% formaldehyde and permeabilization with Permeabilization Medium B (ThermoFisher) for intracellular staining for IFN-γ (clone B27, BD Biosciences) and/or TNF-α (clone MAb11, BD Biosciences). To determine cytokine levels in culture supernatant, T cells (100,000 cells/well) were cocultured with target cells (50,000 cells/well) for 24 hours. Twenty-one of CLDN6 20-mer peptides with 10 amino acid overlapping each was synthesized by EZBiolab. Cancer cells were cultured for 2 days with 1,000 U/ml IFN-γ (PeproTech) and washed thoroughly before the co-culture with T cells. The culture supernatant was harvested, and IFN-γ level was determined using an ELISA kit (eBioscience) according to manufacturer's instruction.

### Tetramer Analysis

HLA-A2-binding epitope of CLDN6 between CLDN6₁₃₁₋₁₄₀ was predicted using SYFPEITHI database. TLIPVCWTA (CLDN6₁₃₂₋₁₄₀) (SEQ ID NO:18) peptide was synthesized by EZBiolab with 82% purity. HLA-A2/peptide monomer was generated with Flex-T HLA-A*02:01 monomer UVX followed by tetramerization using PE-streptavidin according to the manufacturer's instruction (BioLegend). CD8-TCR-or mock-transduced cells were stained with the tetramer followed by anti-CD8 antibody.

### Retroviral Expression of HLA Genes

Coding region of HLA-A/B/C genes were amplified by RT-PCR using a tumor tissue as template and cloned into a murine stem cell virus (MSCV)-based retroviral expression vector. HLA types of cloned genes were determined by Sanger sequencing at the Genomics Shared Resource at Roswell Park. High titer retroviral vectors were produced from PG13 packaging cell lines and were used to transduce K562 and PA-1 cells.

### Statistical Analysis

The statistical difference of CLDN6 expression on EpCAM⁺ and CD45⁺ cells was analyzed by GraphPad Prism software using the two-tailed paired t test.

In specific and illustrative embodiments, the polynucleotide sequences encoding the TCRs of the invention, and the amino acid sequences of the TCR α and TCR β chains encoded by the polynucleotides are as follows, wherein translation initiation and stop codons in the polynucleotide sequences are bold, and artificial mutation for cysteine modification is underlined:
Nucleotide sequences (Initiation and stop codons are shown in Bold; Artificial mutation for cysteine modification is underlined).

(a) CLDN6-specific CD4+ T cell TCR
CD4+ T cell TCR Native alpha chain
>V-GENE and allele Homsap TRAV3*01
>J-GENE and allele Homsap TRAJ9*01
>CDR3 sequence
(SEQ ID NO: 20)
CAVRDIRTGGFKTIF
(SEQ ID NO: 1)
ATGGCCTCTGCACCCATCTCGATGCTTGCGATGCTCTTCACATTGAGTGGGCTGAGAGCT
CAGTCAGTGGCTCAGCCGGAAGATCAGGTCAACGTTGCTGAAGGGAATCCTCTGACTGT
GAAATGCACCTATTCAGTCTCTGGAAACCCTTATCTTTTTTGGTATGTTCAATACCCCAAC
CGAGGCCTCCAGTTCCTTCTGAAATACATCACAGGGGATAACCTGGTTAAAGGCAGCTAT
GGCTTTGAAGCTGAATTTAACAAGAGCCAAACCTCCTTCCACCTGAAGAAACCATCTGCC
CTTGTGAGCGACTCCGCTTTGTACTTCTGTGCTGTGAGAGACATAAGAACTGGAGGCTTC
AAAACTATCTTTGGAGCAGGAACAAGACTATTTGTTAAAGCAAATATCCAGAACCCTGA
CCCTGCCGTGTACCAGCTGAGAGACTCTAAATCCAGTGACAAGTCTGTCTGCCTATTCAC
CGATTTTGATTCTCAAACAAATGTGTCACAAAGTAAGGATTCTGATGTGTATATCACAGA
CAAAACTGTGCTAGACATGAGGTCTATGGACTTCAAGAGCAACAGTGCTGTGGCCTGGA
GCAACAAATCTGACTTTGCATGTGCAAACGCCTTCAACAACAGCATTATTCCAGAAGACA
CCTTCTTCCCCAGCCCAGAAAGTTCCTGTGATGTCAAGCTGGTCGAGAAAAGCTTTGAAA
CAGATACGAACCTAAACTTTCAAAACCTGTCAGTGATTGGGTTCCGAATCCTCCTCCTGA
AAGTGGCCGGGTTTAATCTGCTCATGACGCTGCGGCTGTGGTCCAGCTGA
CD4+ T cell TCR Cysteine-modified alpha chain
(SEQ ID NO: 2)
ATGGCCTCTGCACCCATCTCGATGCTTGCGATGCTCTTCACATTGAGTGGGCTGAGAGCT
CAGTCAGTGGCTCAGCCGGAAGATCAGGTCAACGTTGCTGAAGGGAATCCTCTGACTGT
GAAATGCACCTATTCAGTCTCTGGAAACCCTTATCTTTTTTGGTATGTTCAATACCCCAAC
CGAGGCCTCCAGTTCCTTCTGAAATACATCACAGGGGATAACCTGGTTAAAGGCAGCTAT
GGCTTTGAAGCTGAATTTAACAAGAGCCAAACCTCCTTCCACCTGAAGAAACCATCTGCC
CTTGTGAGCGACTCCGCTTTGTACTTCTGTGCTGTGAGAGACATAAGAACTGGAGGCTTC
AAAACTATCTTTGGAGCAGGAACAAGACTATTTGTTAAAGCAAATATCCAGAACCCTGA
CCCTGCCGTGTACCAGCTGAGAGACTCTAAATCCAGTGACAAGTCTGTCTGCCTATTCAC
CGATTTTGATTCTCAAACAAATGTGTCACAAAGTAAGGATTCTGATGTGTATATCACAGA
CAAATGTGTGCTAGACATGAGGTCTATGGACTTCAAGAGCAACAGTGCTGTGGCCTGGA
GCAACAAATCTGACTTTGCATGTGCAAACGCCTTCAACAACAGCATTATTCCAGAAGACA
CCTTCTTCCCCAGCCCAGAAAGTTCCTGTGATGTCAAGCTGGTCGAGAAAAGCTTTGAAA
CAGATACGAACCTAAACTTTCAAAACCTGTCAGTGATTGGGTTCCGAATCCTCCTCCTGA
AAGTGGCCGGGTTTAATCTGCTCATGACGCTGCGGCTGTGGTCCAGCTGA
CD4+ T cell TCR Native beta chain
>V-GENE and allele Homsap TRBV4-1*01
>J-GENE and allele Homsap TRBJ2-2*01
>D-GENE and allele Homsap TRBD1*01
>CDR3 sequence
(SEQ ID NO: 21)
CASSQGSGQGNTGELFF
(SEQ ID NO: 3)
ATGGGCTGCAGGCTGCTCTGCTGTGCGGTTCTCTGTCTCCTGGGAGCAGTTCCCA
TAGACACTGAAGTTACCCAGACACCAAAACACCTGGTCATGGGAATGACAAATA
AGAAGTCTTTGAAATGTGAACAACATATGGGGCACAGGGCTATGTATTGGTACA
AGCAGAAAGCTAAGAAGCCACCGGAGCTCATGTTTGTCTACAGCTATGAGAAAC
TCTCTATAAATGAAAGTGTGCCAAGTCGCTTCTCACCTGAATGCCCCAACAGCTC
TCTCTTAAACCTTCACCTACACGCCCTGCAGCCAGAAGACTCAGCCCTGTATCTC
TGCGCCAGCAGCCAAGGTAGCGGACAGGGCAACACCGGGGAGCTGTTTTTTGGA
GAAGGCTCTAGGCTGACCGTACTGGAGGACCTGAAAAACGTGTTCCCACCCGAG
GTCGCTGTGTTTGAGCCATCAGAAGCAGAGATCTCCCACACCCAAAAGGCCACA
CTGGTGTGCCTGGCCACAGGCTTCTACCCCGACCACGTGGAGCTGAGCTGGTGGG
TGAATGGGAAGGAGGTGCACAGTGGGGTCAGCACAGACCCGCAGCCCCTCAAGG
AGCAGCCCGCCCTCAATGACTCCAGATACTGCCTGAGCAGCCGCCTGAGGGTCTC
GGCCACCTTCTGGCAGAACCCCCGCAACCACTTCCGCTGTCAAGTCCAGTTCTAC
GGGCTCTCGGAGAATGACGAGTGGACCCAGGATAGGGCCAAACCTGTCACCCAG
ATCGTCAGCGCCGAGGCCTGGGGTAGAGCAGACTGTGGCTTCACCTCCGAGTCTT
ACCAGCAAGGGGTCCTGTCTGCCACCATCCTCTATGAGATCTTGCTAGGGAAGGC
CACCTTGTATGCCGTGCTGGTCAGTGCCCTCGTGCTGATGGCCATGGTCAAGAGA
AAGGATTCCAGAGGCTAG
CD4+ T cell TCR Cysteine-modified beta chain
(SEQ ID NO: 4)
ATGGGCTGCAGGCTGCTCTGCTGTGCGGTTCTCTGTCTCCTGGGAGCAGTTCCCATAGAC
ACTGAAGTTACCCAGACACCAAAACACCTGGTCATGGGAATGACAAATAAGAAGTCTTT
GAAATGTGAACAACATATGGGGCACAGGGCTATGTATTGGTACAAGCAGAAAGCTAAGA
AGCCACCGGAGCTCATGTTTGTCTACAGCTATGAGAAACTCTCTATAAATGAAAGTGTGC
CAAGTCGCTTCTCACCTGAATGCCCCAACAGCTCTCTCTTAAACCTTCACCTACACGCCCT
GCAGCCAGAAGACTCAGCCCTGTATCTCTGCGCCAGCAGCCAAGGTAGCGGACAGGGCA
ACACCGGGGAGCTGTTTTTTGGAGAAGGCTCTAGGCTGACCGTACTGGAGGACCTGAAA
AACGTGTTCCCACCCGAGGTCGCTGTGTTTGAGCCATCAGAAGCAGAGATCTCCCACACC
CAAAAGGCCACACTGGTGTGCCTGGCCACAGGCTTCTACCCCGACCACGTGGAGCTGAG
CTGGTGGGTGAATGGGAAGGAGGTGCACAGTGGGGTCTGCACAGACCCGCAGCCCCTCA
AGGAGCAGCCCGCCCTCAATGACTCCAGATACTGCCTGAGCAGCCGCCTGAGGGTCTCG
GCCACCTTCTGGCAGAACCCCCGCAACCACTTCCGCTGTCAAGTCCAGTTCTACGGGCTC
TCGGAGAATGACGAGTGGACCCAGGATAGGGCCAAACCTGTCACCCAGATCGTCAGCGC
CGAGGCCTGGGGTAGAGCAGACTGTGGCTTCACCTCCGAGTCTTACCAGCAAGGGGTCC
TGTCTGCCACCATCCTCTATGAGATCTTGCTAGGGAAGGCCACCTTGTATGCCGTGCTGG
TCAGTGCCCTCGTGCTGATGGCCATGGTCAAGAGAAAGGATTCCAGAGGCTAG
(b) CLDN6-specific CD8+ T cell TCR
CD8+ T cell TCR Native alpha chain
V-GENE and allele Homsap TRAV21*01
J-GENE and allele Homsap TRAJ9*01
CDR3 sequence
(SEQ ID NO: 22)
CAVMGTGGFKTIF
(SEQ ID NO: 5)
ATGGAGACCCTCTTGGGCCTGCTTATCCTTTGGCTGCAGCTGCAATGGGTGAGCAGCAAA
CAGGAGGTGACGCAGATTCCTGCAGCTCTGAGTGTCCCAGAAGGAGAAAACTTGGTTCT
CAACTGCAGTTTCACTGATAGCGCTATTTACAACCTCCAGTGGTTTAGGCAGGACCCTGG
GAAAGGTCTCACATCTCTGTTGCTTATTCAGTCAAGTCAGAGAGAGCAAACAAGTGGAA
GACTTAATGCCTCGCTGGATAAATCATCAGGACGTAGTACTTTATACATTGCAGCTTCTC
AGCCTGGTGACTCAGCCACCTACCTCTGTGCTGTGATGGGTACTGGAGGCTTCAAAACTA
TCTTTGGAGCAGGAACAAGACTATTTGTTAAAGCAAATATCCAGAACCCTGACCCTGCCG
TGTACCAGCTGAGAGACTCTAAATCCAGTGACAAGTCTGTCTGCCTATTCACCGATTTTG
ATTCTCAAACAAATGTGTCACAAAGTAAGGATTCTGATGTGTATATCACAGACAAAACTG
TGCTAGACATGAGGTCTATGGACTTCAAGAGCAACAGTGCTGTGGCCTGGAGCAACAAA
TCTGACTTTGCATGTGCAAACGCCTTCAACAACAGCATTATTCCAGAAGACACCTTCTTC
CCCAGCCCAGAAAGTTCCTGTGATGTCAAGCTGGTCGAGAAAAGCTTTGAAACAGATAC
GAACCTAAACTTTCAAAACCTGTCAGTGATTGGGTTCCGAATCCTCCTCCTGAAAGTGGC
CGGGTTTAATCTGCTCATGACGCTGCGGCTGTGGTCCAGCTGA
CD8+ T cell TCR Cysteine-modified alpha chain
(SEQ ID NO: 6)
ATGGAGACCCTCTTGGGCCTGCTTATCCTTTGGCTGCAGCTGCAATGGGTGAGCAGCAAA
CAGGAGGTGACGCAGATTCCTGCAGCTCTGAGTGTCCCAGAAGGAGAAAACTTGGTTCT
CAACTGCAGTTTCACTGATAGCGCTATTTACAACCTCCAGTGGTTTAGGCAGGACCCTGG
GAAAGGTCTCACATCTCTGTTGCTTATTCAGTCAAGTCAGAGAGAGCAAACAAGTGGAA
GACTTAATGCCTCGCTGGATAAATCATCAGGACGTAGTACTTTATACATTGCAGCTTCTC
AGCCTGGTGACTCAGCCACCTACCTCTGTGCTGTGATGGGTACTGGAGGCTTCAAAACTA
TCTTTGGAGCAGGAACAAGACTATTTGTTAAAGCAAATATCCAGAACCCTGACCCTGCCG
TGTACCAGCTGAGAGACTCTAAATCCAGTGACAAGTCTGTCTGCCTATTCACCGATTTTG
ATTCTCAAACAAATGTGTCACAAAGTAAGGATTCTGATGTGTATATCACAGACAAATGTG
TGCTAGACATGAGGTCTATGGACTTCAAGAGCAACAGTGCTGTGGCCTGGAGCAACAAA
TCTGACTTTGCATGTGCAAACGCCTTCAACAACAGCATTATTCCAGAAGACACCTTCTTC
CCCAGCCCAGAAAGTTCCTGTGATGTCAAGCTGGTCGAGAAAAGCTTTGAAACAGATAC
GAACCTAAACTTTCAAAACCTGTCAGTGATTGGGTTCCGAATCCTCCTCCTGAAAGTGGC
CGGGTTTAATCTGCTCATGACGCTGCGGCTGTGGTCCAGCTGA
CD8+ T cell TCR Native beta chain
V-GENE and allele Homsap TRBV12-4*01
J-GENE and allele Homsap TRBJ2-7*01
D-GENE and allele Homsap TRBD1*01
CDR3 sequence:
(SEQ ID NO: 23)
CASSFGIYEQYF
(SEQ ID NO: 7)
ATGGGCTCCTGGACCCTCTGCTGTGTGTCCCTTTGCATCCTGGTAGCAAAGCACACAGAT
GCTGGAGTTATCCAGTCACCCCGGCACGAGGTGACAGAGATGGGACAAGAAGTGACTCT
GAGATGTAAACCAATTTCAGGACACGACTACCTTTTCTGGTACAGACAGACCATGATGCG
GGGACTGGAGTTGCTCATTTACTTTAACAACAACGTTCCGATAGATGATTCAGGGATGCC
CGAGGATCGATTCTCAGCTAAGATGCCTAATGCATCATTCTCCACTCTGAAGATCCAGCC
CTCAGAACCCAGGGACTCAGCTGTGTACTTCTGTGCCAGCAGTTTTGGGATATACGAGCA
GTACTTCGGGCCGGGCACCAGGCTCACGGTCACAGAGGACCTGAAAAACGTGTTCCCAC
CCGAGGTCGCTGTGTTTGAGCCATCAGAAGCAGAGATCTCCCACACCCAAAAGGCCACA
CTGGTGTGCCTGGCCACAGGCTTCTACCCCGACCACGTGGAGCTGAGCTGGTGGGTGAAT
GGGAAGGAGGTGCACAGTGGGGTCAGCACAGACCCGCAGCCCCTCAAGGAGCAGCCCG
CCCTCAATGACTCCAGATACTGCCTGAGCAGCCGCCTGAGGGTCTCGGCCACCTTCTGGC
AGAACCCCCGCAACCACTTCCGCTGTCAAGTCCAGTTCTACGGGCTCTCGGAGAATGACG
AGTGGACCCAGGATAGGGCCAAACCTGTCACCCAGATCGTCAGCGCCGAGGCCTGGGGT
AGAGCAGACTGTGGCTTCACCTCCGAGTCTTACCAGCAAGGGGTCCTGTCTGCCACCATC
CTCTATGAGATCTTGCTAGGGAAGGCCACCTTGTATGCCGTGCTGGTCAGTGCCCTCGTG
CTGATGGCCATGGTCAAGAGAAAGGATTCCAGAGGCTAG
CD8+ T cell TCR Cysteine-modified beta chain
(SEQ ID NO: 8)
ATGGGCTCCTGGACCCTCTGCTGTGTGTCCCTTTGCATCCTGGTAGCAAAGCACACAGAT
GCTGGAGTTATCCAGTCACCCCGGCACGAGGTGACAGAGATGGGACAAGAAGTGACTCT
GAGATGTAAACCAATTTCAGGACACGACTACCTTTTCTGGTACAGACAGACCATGATGCG
GGGACTGGAGTTGCTCATTTACTTTAACAACAACGTTCCGATAGATGATTCAGGGATGCC
CGAGGATCGATTCTCAGCTAAGATGCCTAATGCATCATTCTCCACTCTGAAGATCCAGCC
CTCAGAACCCAGGGACTCAGCTGTGTACTTCTGTGCCAGCAGTTTTGGGATATACGAGCA
GTACTTCGGGCCGGGCACCAGGCTCACGGTCACAGAGGACCTGAAAAACGTGTTCCCAC
CCGAGGTCGCTGTGTTTGAGCCATCAGAAGCAGAGATCTCCCACACCCAAAAGGCCACA
CTGGTGTGCCTGGCCACAGGCTTCTACCCCGACCACGTGGAGCTGAGCTGGTGGGTGAAT
GGGAAGGAGGTGCACAGTGGGGTCTGCACAGACCCGCAGCCCCTCAAGGAGCAGCCCGC
CCTCAATGACTCCAGATACTGCCTGAGCAGCCGCCTGAGGGTCTCGGCCACCTTCTGGCA
GAACCCCCGCAACCACTTCCGCTGTCAAGTCCAGTTCTACGGGCTCTCGGAGAATGACGA
GTGGACCCAGGATAGGGCCAAACCTGTCACCCAGATCGTCAGCGCCGAGGCCTGGGGTA
GAGCAGACTGTGGCTTCACCTCCGAGTCTTACCAGCAAGGGGTCCTGTCTGCCACCATCC
TCTATGAGATCTTGCTAGGGAAGGCCACCTTGTATGCCGTGCTGGTCAGTGCCCTCGTGC
TGATGGCCATGGTCAAGAGAAAGGATTCCAGAGGCTAG
Amino acid sequences (Stop codon is shown as asterisk (*), CDR3
sequence is in Bold, Artificial cysteine modification is underlined).
(a) CLDN6-specific CD4+ T cell TCR
CD4+ T cell TCR Native alpha chain
(SEQ ID NO: 9)
MASAPISMLAMLFTLSGLRAQSVAQPEDQVNVAEGNPLTVKCTYSVSGNPYLFWYVQYPNR
GLQFLLKYITGDNLVKGSYGFEAEFNKSQTSFHLKKPSALVSDSALYFCAVRDIRTGGFKTI
FGAGTRLFVKANIQNPDPAVYQLRDSKSSDKSVCLFTDFDSQTNVSQSKDSDVYITDKTVLD
MRSMDFKSNSAVAWSNKSDFACANAFNNSIIPEDTFFPSPESSCDVKLVEKSFETDTNLNFQN
LSVIGFRILLLKVAGFNLLMTLRLWSS*
CD4+ T cell TCR Cysteine-modified alpha chain
(SEQ ID NO: 10)
MASAPISMLAMLFTLSGLRAQSVAQPEDQVNVAEGNPLTVKCTYSVSGNPYLFWYVQYPNR
GLQFLLKYITGDNLVKGSYGFEAEFNKSQTSFHLKKPSALVSDSALYFCAVRDIRTGGFKTI
FGAGTRLFVKANIQNPDPAVYQLRDSKSSDKSVCLFTDFDSQTNVSQSKDSDVYITDKCVLD
MRSMDFKSNSAVAWSNKSDFACANAFNNSIIPEDTFFPSPESSCDVKLVEKSFETDTNLNFQN
LSVIGFRILLLKVAGFNLLMTLRLWSS*
CD4+ T cell TCR Native beta chain
(SEQ ID NO: 11)
MGCRLLCCAVLCLLGAVPIDTEVTQTPKHLVMGMTNKKSLKCEQHMGHRAMYWYKQKAK
KPPELMFVYSYEKLSINESVPSRFSPECPNSSLLNLHLHALQPEDSALYLCASSQGSGQGNTG
ELFFGEGSRLTVLEDLKNVFPPEVAVFEPSEAEISHTQKATLVCLATGFYPDHVELSWWVNG
KEVHSGVSTDPQPLKEQPALNDSRYCLSSRLRVSATFWQNPRNHFRCQVQFYGLSENDEWT
QDRAKPVTQIVSAEAWGRADCGFTSESYQQGVLSATILYEILLGKATLYAVLVSALVLMAM
VKRKDSRG*
CD4+ T cell TCR Cysteine-modified beta chain
(SEQ ID NO: 12)
MGCRLLCCAVLCLLGAVPIDTEVTQTPKHLVMGMTNKKSLKCEQHMGHRAMYWYKQKAK
KPPELMFVYSYEKLSINESVPSRFSPECPNSSLLNLHLHALQPEDSALYLCASSQGSGQGNTG
ELFFGEGSRLTVLEDLKNVFPPEVAVFEPSEAEISHTQKATLVCLATGFYPDHVELSWWVNG
KEVHSGVCTDPQPLKEQPALNDSRYCLSSRLRVSATFWQNPRNHFRCQVQFYGLSENDEWT
QDRAKPVTQIVSAEAWGRADCGFTSESYQQGVLSATILYEILLGKATLYAVLVSALVLMAM
VKRKDSRG*
(b) CLDN6-specific CD8+ T cell TCR
CD8+ T cell TCR Native alpha chain
(SEQ ID NO: 13)
METLLGLLILWLQLQWVSSKQEVTQIPAALSVPEGENLVLNCSFTDSAIYNLQWFRQDPGKG
LTSLLLIQSSQREQTSGRLNASLDKSSGRSTLYIAASQPGDSATYLCAVMGTGGFKTIFGAGT
RLFVKANIQNPDPAVYQLRDSKSSDKSVCLFTDFDSQTNVSQSKDSDVYITDKTVLDMRSMD
FKSNSAVAWSNKSDFACANAFNNSIIPEDTFFPSPESSCDVKLVEKSFETDTNLNFQNLSVIGF
RILLLKVAGFNLLMTLRLWSS*
CD8+ T cell TCR Cysteine-modified alpha chain
(SEQ ID NO: 14)
METLLGLLILWLQLQWVSSKQEVTQIPAALSVPEGENLVLNCSFTDSAIYNLQWFRQDPGKG
LTSLLLIQSSQREQTSGRLNASLDKSSGRSTLYIAASQPGDSATYLCAVMGTGGFKTIFGAGT
RLFVKANIQNPDPAVYQLRDSKSSDKSVCLFTDFDSQTNVSQSKDSDVYITDKCVLDMRSMD
FKSNSAVAWSNKSDFACANAFNNSIIPEDTFFPSPESSCDVKLVEKSFETDTNLNFQNLSVIGF
RILLLKVAGFNLLMTLRLWSS*
CD8+ T cell TCR Native beta chain
(SEQ ID NO: 15)
MGSWTLCCVSLCILVAKHTDAGVIQSPRHEVTEMGQEVTLRCKPISGHDYLFWYRQTMMRG
LELLIYFNNNVPIDDSGMPEDRFSAKMPNASFSTLKIQPSEPRDSAVYFCASSFGIYEQYFGPG
TRLTVTEDLKNVFPPEVAVFEPSEAEISHTQKATLVCLATGFYPDHVELSWWVNGKEVHSGV
STDPQPLKEQPALNDSRYCLSSRLRVSATFWQNPRNHFRCQVQFYGLSENDEWTQDRAKPV
TQIVSAEAWGRADCGFTSESYQQGVLSATILYEILLGKATLYAVLVSALVLMAMVKRKDSR
G*
CD8+ T cell TCR Cysteine-modified beta chain
(SEQ ID NO: 16)
MGSWTLCCVSLCILVAKHTDAGVIQSPRHEVTEMGQEVTLRCKPISGHDYLFWYRQTMMRG
LELLIYFNNNVPIDDSGMPEDRFSAKMPNASFSTLKIQPSEPRDSAVYFCASSFGIYEQYFGPG
TRLTVTEDLKNVFPPEVAVFEPSEAEISHTQKATLVCLATGFYPDHVELSWWVNGKEVHSGV
CTDPQPLKEQPALNDSRYCLSSRLRVSATFWQNPRNHFRCQVQFYGLSENDEWTQDRAKPV
TQIVSAEAWGRADCGFTSESYQQGVLSATILYEILLGKATLYAVLVSALVLMAMVKRKDSR
G*

### Results

### Expression of CLDN6 on EpCAM+ Cancer Cells in Ascites of Ovarian Cancer Patients

To investigate cell surface CLDN6 expression on primary ovarian cancer cells, mononuclear cells from ascites of 32 ovarian cancer patients were stained with antibodies for CLDN6, EpCAM and CD45, and analyzed by flow cytometry (FIG. 1). CLDN6 expression on EpCAM⁺ and CD45⁺ cells was determined using a Fluorescence Minus One (FMO) control (FIG. 1A). CLDN6 was significantly but variably expressed on EpCAM⁺ cells but not on CD45⁺ cells (FIG. 1B). Mean frequency of CLDN6 expression on EpCAM⁺ cells was 30% (range 0.2%-80.0%) while it was 0.17% on CD45⁺ cells (0%-3.0%). CLDN6 expression on EpCAM⁻CD45⁻ cells was negligible (data not shown). The lack of expression in normal tissues, but frequent and tumor -specific expression suggest that CLDN6 is a promising target for immunotherapy for ovarian cancer patients

### Detection of Spontaneous T-Cell Responses Against CLDN6 in Ovarian Cancer Patients

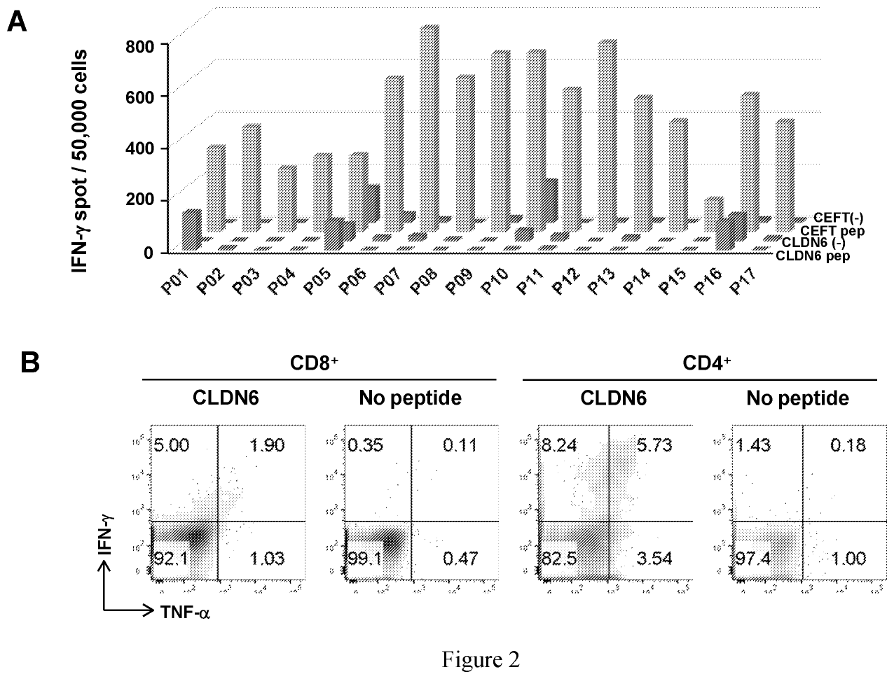

To investigate spontaneously induced T-cell response against CLDN6 in ovarian cancer patients, CLDN6-specific response was tested following in vitro stimulation of PBMC with a pool of overlapping peptides. One (P01) out of 17 patients showed specific response against CLDN6 based on the criteria of positive response (the number of spots is >50 and twice higher than background) (FIG. 2A). For the patient P01, we found that both CD8⁺ and CD4⁺ T cells were reactive to CLDN6 peptides by intracellular cytokine staining (FIG. 2B)

### Generation of CLDN6-Specific T Cells by TCR Gene-Engineering

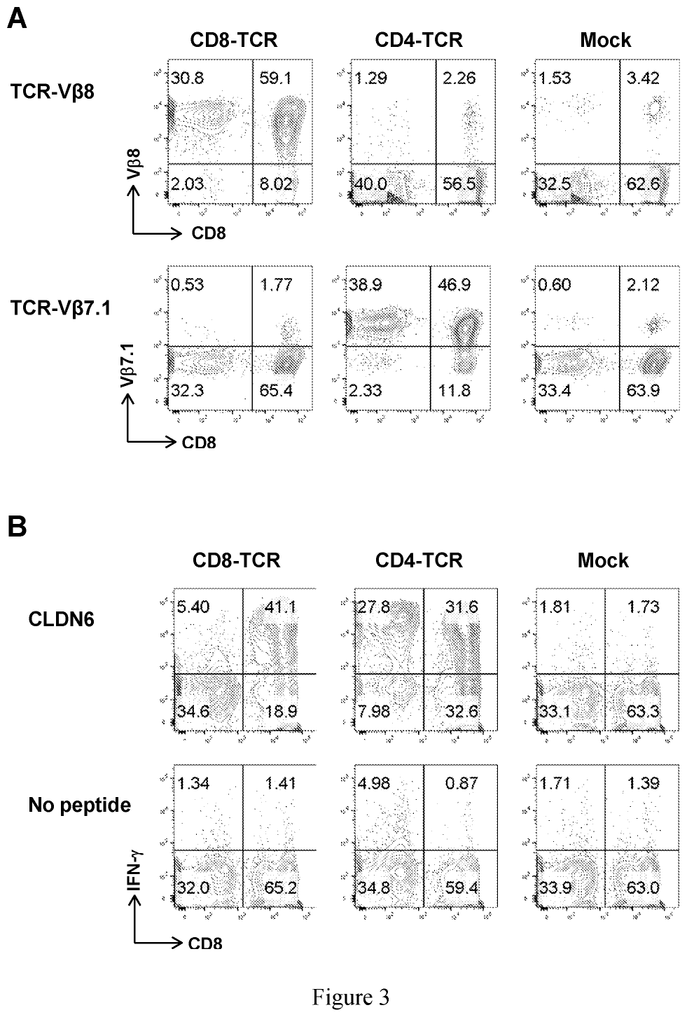

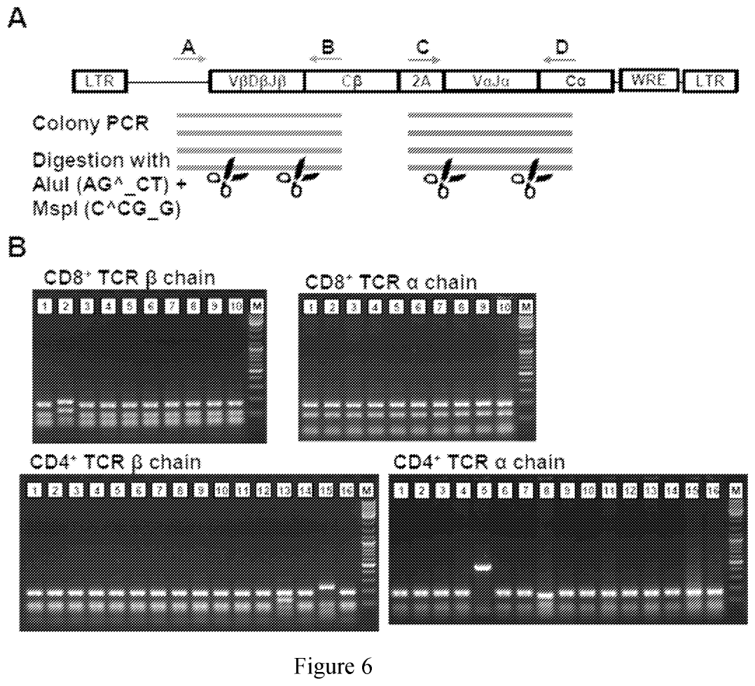

To clone TCR genes, CLDN6-specific CD4⁺ and CD8⁺ T cells were isolated by sorting IFN-γ-producing cells after co-culture with CLDN6 peptide-pulsed autologous antigen-presenting cells. The coding region of TCR α and β chain genes that was amplified by RT-PCR from sorted CD8⁺ or CD4⁺ T cells was assembled as an expression cassette in a retroviral plasmid vector by our TCR-expressing retroviral vector construction method. DNA fingerprinting of TCR inserts in plasmid clones by restriction enzyme digestion showed that the majority of plasmid clones contained the same TCR inserts, indicating a monoclonal CD4⁺ and CD8⁺ T-cell response against CLDN6 (FIG. 6). Retroviral particles produced from PG13 virus-producing cells were used to transduce TCR genes to T cells derived from healthy donor PBMC. According to the Sanger sequencing data of TCR-expressing plasmids, we identified TCR Vβ subtype of CLDN6-specific CD8⁺ T cells and CD4⁺ T cells as Vβ8 and Vβ7.1, respectively. TCR-transduced T cells were stained with those TCR Vβ antibodies and the transduction efficiency was >85% (FIG. 3A). The reactivity of TCR-transduced T cells to CLDN6 peptide was confirmed by intracellular cytokine staining (FIG. 3B). While both CD8⁺ and CD8⁻ (i.e. CD4⁺) T cells transduced with TCR derived from CD4⁺ T cells (CD4-TCR) strongly produced IFN-γ against peptides, CD8⁻ T cells transduced with CD8-TCR showed a weak response, indicating that CD8-TCR requires CD8 co-ligation for the recognition.

Identification of Epitope and HLA Restriction for CLDN6-Specific CD8+ and CD4+ T Cells

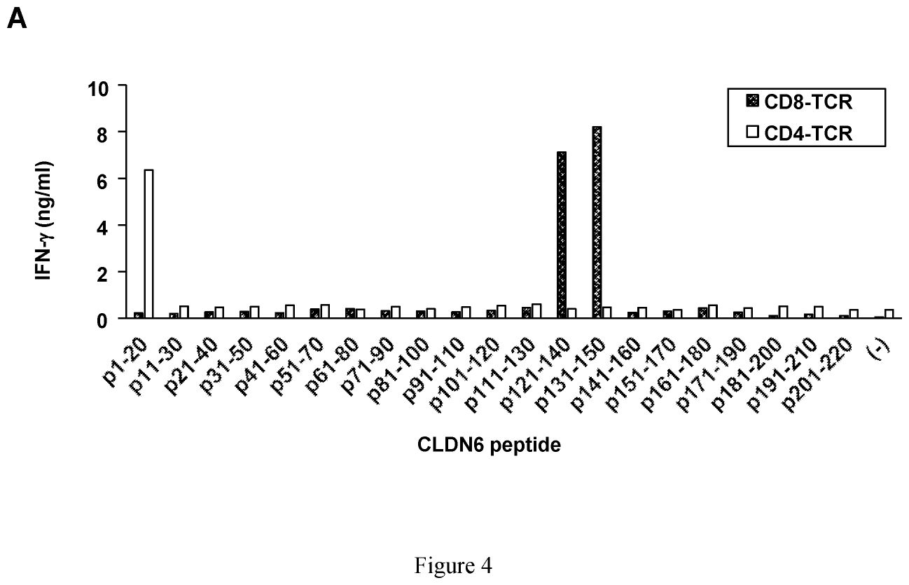

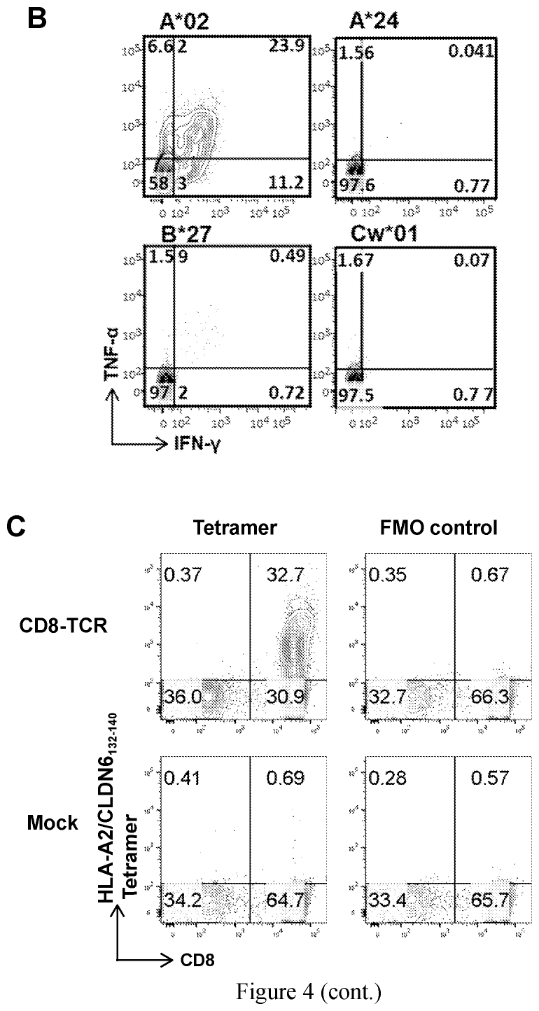

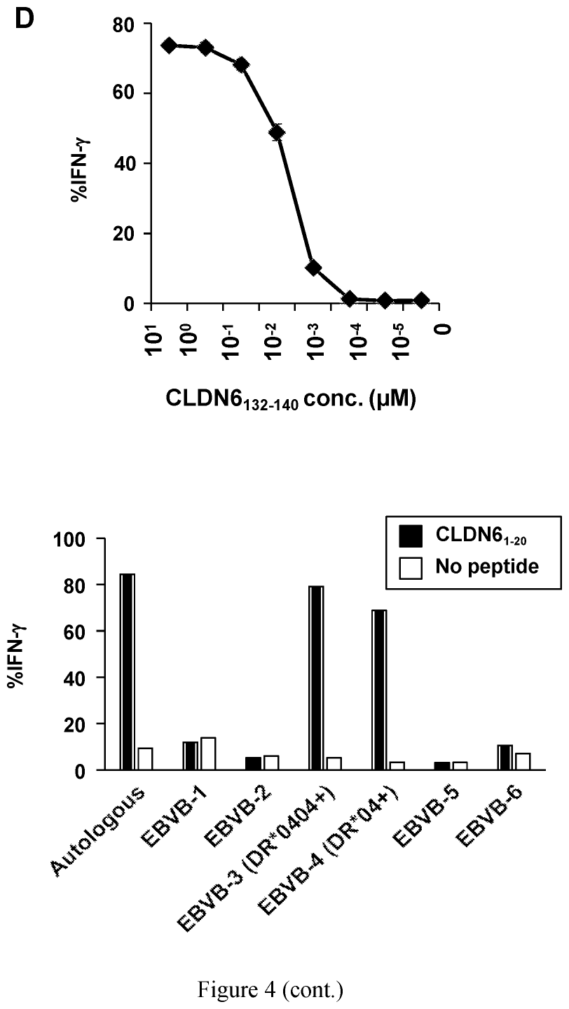

By utilizing TCR-transduced T cells, we characterized a peptide region recognized by CLDN6-specific T cells. By determining reactivity of CD8-TCR-and CD4-TCR-transduced T cells against individual peptides in the CLDN6 peptide pool, the epitope for CD4-TCR was determined to be in the N-terminal CLDN6₁₋₂₀ peptide in the signal peptide region while the epitope for CD8-TCR is in the overlapping region of peptides CLDN6₁₂₁₋₁₄₀ and CLDN6₁₃₁₋₁₅₀, as CD8-TCR-transduced T cells similarly recognized these peptides (FIG. 4A). HLA restriction of CD8-TCR was determined by using K562 cells that were transduced with the patient P01's HLA class I. HLA class I types of the patient P01 were determined to be HLA-A2,-A*24:02,-B*27 and -Cw*01. HLA-negative K562 cells were retrovirally transduced with these HLA genes, pulsed with CLDN6 peptides, and co-cultured with CD8-TCR-transduced T cells. CD8-TCR was found to be restricted to HLA-A2 as it showed strong reactivity against peptides only when pulsed on HLA-A2-transduced K562 cells (FIG. 4B). Based on HLA-A2 and peptide region recognized by CD8-TCR, HLA-A2-restricted epitope of CLDN6 was predicted as TLIPVCWTA (CLDN6₁₃₂₋₁₄₀) (SEQ ID NO: 18) using the SYFPEITHI algorithm. TCR binding of the predicted epitope was demonstrated by HLA-A2/CLDN6₁₃₂₋₁₄₀ tetramer staining (FIG. 4C). Using the CLDN6₁₃₂₋₁₄₀ peptide, we tested TCR avidity against the peptide and found that CD8-TCR-transduced T cells efficiently recognized 1 nM peptide (FIG. 4D). HLA restrictions of CD4-TCR were identified as HLA-DR*04:04 based on the peptide reactivity using a panel of partially HLA-matched EBV-B cells as antigen-presenting cells (FIG. 4E and Table 1)

### Cancer Cell Recognition by CD8-TCR-Transduced T Cells

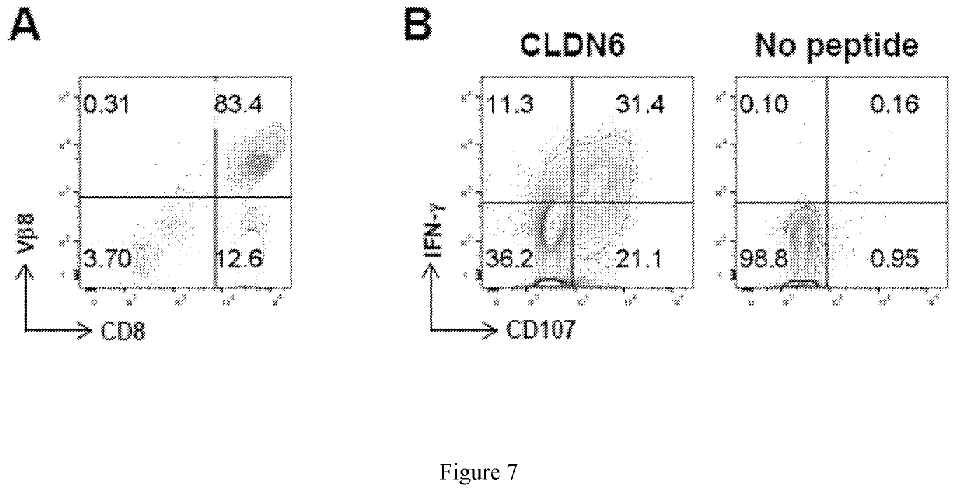

We next assessed the reactivity of CD8-TCR-transduced T cells against CLDN6⁺ ovarian cancer cells. To compare the reactivity of TCR-transduced T cells with that of naturally occurring CLDN6-specific CD8⁺ T cells, a part of sorted CLDN6-specific CD8⁺ T cells was polyclonally expanded. As expected, CLDN6-specific CD8⁺ T-cell line expressed TCR Vβ8 and showed strong reactivity against peptide-pulsed target cells (FIGS. 7A and 7B).

We investigated expression of CLDN6 and HLA-A2 on the patient P01-derived EOC cell line and an established ovarian cancer cell line PA-1 which was reported to highly express CLDN6 by flow cytometry. As PA-1 cells is HLA-A2 negative, HLA-A2 was retrovirally transduced. We found that both ovarian cancer cell lines express CLDN6 (FIG. 5A). Although the patient P01 was HLA-A2⁺, the autologous EOC cell line showed marginal cell surface HLA-A2 expression (FIG. 5A). To upregulate HLA expression, the cancer cells were treated with IFN-γ. IFN-γ treatment upregulated HLA-A2 on the autologous EOC and HLA-A2-transduced PA-1 cells but did not affect the intensity of CLDN6 expression (FIG. 5A). When T cells were cocultured with these cancer cells, parental CD8⁺ T-cell line recognized EOC cells only after treatment with IFN-γ (FIG. 5B). CD8-TCR transduced T cells recognized IFN-γ-treated autologous EOC cells with similar efficiency to the parental CD8+ T-cell line. CD8-TCR-transduced T cells also showed strong reactivity against HLA-A2-transduced and IFN-γ-treated PA-1 cells (FIG. 5C). The results indicate that CD8-TCR transduced T cells can recognize cancer cells expressing CLDN6 and HLA-A2.

Although the invention has been described in detail for the purposes of illustration, it is understood that such detail is solely for that purpose, and variations can be made therein by those skilled in the art without departing from the spirit and scope of the invention which is defined by the following claims.

1. An expression vector encoding an alpha chain and a beta chain of a T cell receptor (TCR), wherein T cells modified to comprise the expression vector and express the alpha and beta chains of the TCR specifically recognize a Claudin 6 (CLDN6) protein epitope in a human leukocyte antigen (HLA) context.

2. The expression vector of claim 1, wherein the alpha chain comprises the sequence of SEQ ID NO:9, wherein optionally the 182nd amino acid is Cysteine, and the beta chain comprises the sequence of SEQ ID NO:11, wherein optionally the 192nd amino acid is Cysteine.

3. The expression vector of claim 2, wherein the T cells are CD4+ T cells.

4. The expression vector of claim 1, wherein the alpha chain comprises the sequence of SEQ ID NO:13, wherein optionally the 179th amino acid is Cysteine, and a beta chain having the sequence of SEQ ID NO:15, wherein optionally the 188th amino acid is Cysteine.

5. The expression vector of claim 4, wherein the T cells are CD8+ T cells.

6. The expression vector of claim 3, wherein the alpha chain, the beta chain, or both, comprise said Cysteine.

7. A hematopoietic stem cell or a T cell comprising an expression vector as in claim 1.

8. A method comprising administering to an individual who has a CLDN6 positive cancer a population of hematopoietic stem cells or T cells as in claim 7 to thereby inhibit the growth of the cancer and/or kill the cancer cells.

9. The method of claim 8, wherein the population of T cells comprises CD8+ T cells, CD4+ T cells, or a combination thereof.

10. The method of claim 9, wherein the population of T cells comprises the CD8+ T cells.

11. The method of claim 9, wherein the population of T cells comprises the CD4+ T cells.

12. The method of claim 9, wherein the population of T cells comprises the combination of CD4+ and the CD8+ T cells.

13. The method of claim 8, wherein the CLDN6 positive cancer is ovarian cancer.

14. The method of claim 9, wherein the CLDN6 positive cancer is ovarian cancer.

15. The method of claim 10, wherein the CLDN6 positive cancer is ovarian cancer.

16. The method of claim 11, wherein the CLDN6 positive cancer is ovarian cancer.

17. The method of claim 12, wherein the CLDN6 positive cancer is ovarian cancer.

18. A method of making a modified T cell or a modified hematopoietic stem cell, the method comprising introducing into a T cell or a hematopoietic stem cell an expression vector as in claim 1 to thereby produce the modified T cell or the modified hematopoietic stem cell.

19. The method of claim 18, the method comprising making the modified T cell.

20. The method of claim 19, wherein the modified T cell is a CD4+ or CD8+ T cell.


SEQUENCE LISTING (23 sequences)

SEQ ID NO: 1 - DNA, 828 bp, Homo sapiens
  source 1..828: mol_type = other DNA
atggcctctgcacccatctcgatgcttgcgatgctcttcacattgagtgggctgagagctcagtcagtggctcagccggaagatcaggtcaacgttgctgaagggaatcctctgactgtgaaatgcacctattcagtctctggaaacccttatcttttttggtatgttcaataccccaaccgaggcctccagttccttctgaaatacatcacaggggataacctggttaaaggcagctatggctttgaagctgaatttaacaagagccaaacctccttccacctgaagaaaccatctgcccttgtgagcgactccgctttgtacttctgtgctgtgagagacataagaactggaggcttcaaaactatctttggagcaggaacaagactatttgttaaagcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 2 - DNA, 828 bp, synthetic construct
  source 1..828: mol_type = other DNA
atggcctctgcacccatctcgatgcttgcgatgctcttcacattgagtgggctgagagctcagtcagtggctcagccggaagatcaggtcaacgttgctgaagggaatcctctgactgtgaaatgcacctattcagtctctggaaacccttatcttttttggtatgttcaataccccaaccgaggcctccagttccttctgaaatacatcacaggggataacctggttaaaggcagctatggctttgaagctgaatttaacaagagccaaacctccttccacctgaagaaaccatctgcccttgtgagcgactccgctttgtacttctgtgctgtgagagacataagaactggaggcttcaaaactatctttggagcaggaacaagactatttgttaaagcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 3 - DNA, 945 bp, Homo sapiens
  source 1..945: mol_type = other DNA
atgggctgcaggctgctctgctgtgcggttctctgtctcctgggagcagttcccatagacactgaagttacccagacaccaaaacacctggtcatgggaatgacaaataagaagtctttgaaatgtgaacaacatatggggcacagggctatgtattggtacaagcagaaagctaagaagccaccggagctcatgtttgtctacagctatgagaaactctctataaatgaaagtgtgccaagtcgcttctcacctgaatgccccaacagctctctcttaaaccttcacctacacgccctgcagccagaagactcagccctgtatctctgcgccagcagccaaggtagcggacagggcaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 4 - DNA, 945 bp, synthetic construct
  source 1..945: mol_type = other DNA
atgggctgcaggctgctctgctgtgcggttctctgtctcctgggagcagttcccatagacactgaagttacccagacaccaaaacacctggtcatgggaatgacaaataagaagtctttgaaatgtgaacaacatatggggcacagggctatgtattggtacaagcagaaagctaagaagccaccggagctcatgtttgtctacagctatgagaaactctctataaatgaaagtgtgccaagtcgcttctcacctgaatgccccaacagctctctcttaaaccttcacctacacgccctgcagccagaagactcagccctgtatctctgcgccagcagccaaggtagcggacagggcaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 5 - DNA, 819 bp, Homo sapiens
  source 1..819: mol_type = other DNA
atggagaccctcttgggcctgcttatcctttggctgcagctgcaatgggtgagcagcaaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgatgggtactggaggcttcaaaactatctttggagcaggaacaagactatttgttaaagcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 6 - DNA, 819 bp, synthetic construct
  source 1..819: mol_type = other DNA
atggagaccctcttgggcctgcttatcctttggctgcagctgcaatgggtgagcagcaaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgatgggtactggaggcttcaaaactatctttggagcaggaacaagactatttgttaaagcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 7 - DNA, 933 bp, Homo sapiens
  source 1..933: mol_type = other DNA
atgggctcctggaccctctgctgtgtgtccctttgcatcctggtagcaaagcacacagatgctggagttatccagtcaccccggcacgaggtgacagagatgggacaagaagtgactctgagatgtaaaccaatttcaggacacgactaccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagttttgggatatacgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 8 - DNA, 933 bp, synthetic construct
  source 1..933: mol_type = other DNA
atgggctcctggaccctctgctgtgtgtccctttgcatcctggtagcaaagcacacagatgctggagttatccagtcaccccggcacgaggtgacagagatgggacaagaagtgactctgagatgtaaaccaatttcaggacacgactaccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagttttgggatatacgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 9 - protein, 275 aa, Homo sapiens
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDIRTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 10 - protein, 275 aa, synthetic construct
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDIRTGGFKTIFGAGTRLFVKANIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 11 - protein, 314 aa, Homo sapiens
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSQGSGQGNTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 12 - protein, 314 aa, synthetic construct
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSQGSGQGNTGELFFGEGSRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 13 - protein, 272 aa, Homo sapiens
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVMGTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 14 - protein, 272 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVMGTGGFKTIFGAGTRLFVKANIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 15 - protein, 310 aa, Homo sapiens
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSFGIYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 16 - protein, 310 aa, synthetic construct
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSFGIYEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 17 - protein, 220 aa, Homo sapiens
MASAGMQILGVVLTLLGWVNGLVSCALPMWKVTAFIGNSIVVAQVVWEGLWMSCVVQSTGQMQCKVYDSLLALPQDLQAARALCVIALLVALFGLLVYLAGAKCTTCVEEKDSKARLVLTSGIVFVISGVLTLIPVCWTAHAIIRDFYNPLVAEAQKRELGASLYLGWAASGLLLLGGGLLCCTCPSGGSQGPSHYMARYSTSAPAISRGPSEYPTKNYV

SEQ ID NO: 18 - protein, 9 aa, Homo sapiens
TLIPVCWTA

SEQ ID NO: 19 - protein, 20 aa, Homo sapiens
MASAGMQILGVVLTLLGWVN

SEQ ID NO: 20 - protein, 15 aa, Homo sapiens
CAVRDIRTGGFKTIF

SEQ ID NO: 21 - protein, 17 aa, Homo sapiens
CASSQGSGQGNTGELFF

SEQ ID NO: 22 - protein, 13 aa, Homo sapiens
CAVMGTGGFKTIF

SEQ ID NO: 23 - protein, 12 aa, Homo sapiens
CASSFGIYEQYF